(19) United States
(12) Patent Application Publication
(10) Pub. No.: US 2018/0295931 A1
(43) Pub. Date: Oct. 18, 2018

(54) CORRECTIVE INSOLE

(71) Applicant: KWAN HO SHIN, DAEGU (KR)

(72) Inventor: KWAN HO SHIN, DAEGU (KR)

(21) Appl. No.: 15/952,238

(22) Filed: Apr. 13, 2018

(30) Foreign Application Priority Data
Apr. 14, 2017 (KR) 10-2017-0048594

(51) Int. Cl. A43B 7/14; A43B 7/24; A43B 17/00; A61F 5/14

(52) U.S. Cl. CPC A43B 7/141; A43B 7/1435; A43B 7/149; A61F 5/14; A43B 7/143; A43B 17/00; A43B 7/24

(57) ABSTRACT

A corrective insole having a bottom part including a posterior molded part formed at the position corresponding to the calcaneus, an anterior height part and a skinned part. The anterior height part is formed at a position corresponding to the head of the fifth metatarsal bone. The corrective insole is configured to have a minimal difference in height between the tip and the heel of the corrective insole or the tip is slightly higher than the heel to shift the wearer's body weight to the back when the wearer is in a standing position. The skinned part has a boundary protrusion part, which is formed between the posterior molded part and the anterior height part, configured to shift the wearer's body weight to the outside and scatter the wearer's body weight along the lateral side of the corrective insole.

### BACKGROUND OF THE INVENTION

Field of the Invention

The present invention relates to a corrective insole, and more particularly, to a corrective insole having a bottom part, which includes a posterior molded part formed at the position corresponding to the calcaneus, an anterior height part formed at the position corresponding to the head of the fifth metatarsal bone, and a skinned part having a boundary protrusion part formed between the posterior molded part and the anterior height part, thereby making weight shifted to the outside and scattering the weight along the most lateral side of the corrective insole.

Background Art

The sole of a human body consists of two important arches: one being a lateral plantar arch for supporting the weight of the body; and the other being a medial plantar arch functioning as a spring during an exercise.

The lateral plantar arch includes the calcaneus which is the heel, the cuboid bone which is the most lateral of the bones, and the fifth metatarsal.

The medial plantar arch includes the calcaneus, the talus, the navicular, the cuneiform which is an arch part, and the first metatarsal bone.

However, the feet of the human body become flat feet due to exercises on a hard ground, sudden exercises, lack of flexibility in crural muscles, and so on.

Because weight does not move to the lateral plantar arch while the weight moves from the outside to the inside in an arch form, load of the medial plantar arch cumulates, hence the arch form of the medial plantar arch is collapsed and the feet become flat feet. So, while a person walks for a long time, the person has pain in the front part of the feet, namely, in the metatarsal bone and the phalanges.

So, the person may suffer from a foot disease, such as plantar fasciitis, and the pain may be transferred to the ankle, the knee, the pelvis and the waist in order because the legs turn into knock-kneed legs or bowed legs which are called pronation.

That is, the human body has several problems due to the collapse of the scattering principle of power.

Now, there are a correction and a surgery for treating flat feet, and most of people tend to use correction because surgeries are complicated in process and may cause aftereffects.

Conventional corrective insoles have been manufactured in such a way to make the heel higher and the arch higher without regard to scattering of power in the foot.

So, the conventional corrective insoles just function to support the collapsed arch regardless of movement of weight.

The heel is the first part to support the weight of the human body and is a part to support the heaviest weight. However, regardless of such features of the heel, to simply make the heel higher and the arch of the foot higher just is equal to wear high heels.

If the weight is loaded forward, the knees drives forward, the back is crooked and the neck goes forward in order to maintain stability of the body. So, it causes abnormal walking and pains in knees, the cervical vertebral, the thoracic vertebrae, and the lumbar.

The conventional corrective insoles cause a continuous pain in ligaments and muscles because they support and lift up the arches, so it causes more stress on the feet.

Moreover, the foot frame of a human body evolved the most effectively to walk on plains. The foot of the human body consists of 33 joints, 28 bones, and 44 small and large muscles and tendons, so they make their peculiar activities when the person walks.

As shown in FIG. 1, bones of the foot include the calcaneus, the talus, the navicular bone, the cuboid bone, the cuneiform, the metatarsal bone, the phalanx, and so on.

The foot has four pedal arches. Out of them, the medial plantar arch which exists on the inward side of the foot, and the lateral plantar arch which exists on the outward side of the foot are important.

Bones of the medial plantar arch are the calcaneus, the talus, the navicular, the cuneiform, and the first metatarsal bone.

Furthermore, bones of the lateral plantar arch are the calcaneus, the cuboid, and the fifth metatarsal bone.

The medial plantar arch functions to reduce impact from the weight of the body. Additionally, when the sole of the foot completely touches the ground during walking, namely, when the weight of the body is put on one leg, the tibia turns in and the anklebone directs inward in the ankle joint so that the medial plantar arch subsides. For instance, the medial plantar arch functions as a spring.

However, if the medial plantar arch hyper-pronates, it causes various orthopedic lower extremity diseases. As a representative disease, there is a pronation distortion syndrome. The pronation distortion syndrome means an internal rotation of the knees and adduction, namely, knock-kneed legs, which entail super-pronation of the feet or flat feet.

Such super-pronation causes imbalance in muscles that causes pains in the feet, the ankles, the knees, the hips, and the waist, and increases a danger of wounds in soft tissues, such as the Achilles tendon, arches of feet, the kneepan tendon, and the iliotibial bands, because it cumulates internal rotation stress on the pelvis and the lower part of the waist by causing a change in alignment of the pelvis.

Furthermore, it gives pressure to the subtalar joint, the kneepan joint, the tibiofemoral joint, the iliofemoral joint, and the acroiliac articulation, and it causes anteversion of the pelvis.

The pronation of the feet at 2° or 3° causes 20% to 30% pelvic tilt when the person stands up and causes 50% to 70% anterior pelvic tilt when the person walks, so it causes imbalance in the waist and the vertebrae.

### PATENT LITERATURE

### Patent Documents

Patent Document 1: Korean Utility Model Registration No. 20-0448371

### SUMMARY OF THE INVENTION

Accordingly, the present invention has been made in view of the above-mentioned problems occurring in the prior art, and it is an object of the present invention to provide a corrective insole, which can more stably support weight of a human body on the heel and make the weight of the body move along the normal foot pressure walking pattern when a person walks.

To accomplish the above object, according to the present invention, there is provided a corrective insole having a bottom part including: a posterior molded part formed at the position corresponding to the calcaneus; an anterior height part formed at the position corresponding to the head of the fifth metatarsal bone; and a skinned part having a boundary protrusion part, which is formed between the posterior molded part and the anterior height part.

Moreover, there is little difference in height between the tip and the heel or the tip is slightly higher than the heel.

The skinned part is configured to make weight shifted to the outside so as to scatter the weight along the most lateral side of the corrective insole, and the difference in height between the tip and the heel is formed in such a way that 60% to 80& of the weight of the body is scattered to the heel and 20% to 40% is scattered to the tip, so that the optimum correction effect is provided when the wearer wears shoes of which the heel is 1 cm to 3 cm higher than the tip having the corrective insole.

The corrective insole according to the present invention can make a load by weight of the body and a load by an exercise shifted on the heel of the foot when the wearer stands up, and receives a load transferred from the calcaneus which is the heel of the foot and transfers the received load to the cuboid bone which is the most lateral of the bones of the foot, so as to optimally scatter the loads transferred to the foot.

Therefore, the corrective insole according to the present invention can prevent foot diseases and pronation, such as knock-kneed legs or bowed legs, and makes the load of the knee transferred from the anterior cartilage to the posterior cartilage through correction so that the pelvis, the back and the neck are straightened naturally.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a view showing a structure of bones of the foot;

FIG. 2 is a perspective view showing a base side of a corrective insole according to a first preferred embodiment of the present invention;

FIG. 3 is a front view showing the base side of the corrective insole according to the first preferred embodiment of the present invention;

FIG. 4 is a schematic perspective view showing a used state of the corrective insole according to the first preferred embodiment of the present invention;

FIG. 5 is a perspective view showing a base side of a corrective insole according to a second preferred embodiment of the present invention;

FIG. 6 is a front view showing the base side of the corrective insole according to the second preferred embodiment of the present invention;

FIG. 7 is a schematic perspective view showing a used state of the corrective insole according to the second preferred embodiment of the present invention;

FIG. 8 is a perspective view showing a used state of the corrective insole of the present invention; and

FIG. 9 is a front view showing a difference in height of the tip and the heel of the corrective insole according to the present invention.

### DETAILED DESCRIPTION OF THE PREFERRED EMBODIMENT

Hereinafter, example embodiments will be described in detail with reference to the accompanying drawings so that inventive concept may be readily implemented by those skilled in the art. However, it is to be noted that the present disclosure is not limited to the example embodiments but can be realized in various other ways.

In the drawings, certain parts not directly relevant to the description are omitted to enhance the clarity of the drawings, and like reference numerals denote like parts throughout the whole document.

Throughout the whole specification, unless explicitly stated to the contrary, the word “comprise,” “comprises” or “comprising” used throughout the specification will not be understood as the exclusion of the other elements but to imply the inclusion of the other elements.

First, a bottom part 30 of a corrective insole 10 according to a first preferred embodiment of the present invention has a skinned part 80 formed from the calcaneus to the head of the fifth metatarsal bone so that the weight of a human body can be easily transferred to the most lateral side 90 and can be scattered along the most lateral side 90.

Here, the bottom part 30 of the corrective insole 10 includes a posterior molded part 60 formed at the position corresponding to the calcaneus, and an anterior height part 50 formed at the position corresponding to the head of the fifth metatarsal bone.

Here, the skinned part 80 having a boundary protrusion part 70 is formed between the posterior molded part 60 and the anterior height part 50.

Moreover, the skinned part 80 makes weight shifted to the outside to scatter the weight along the most lateral side 90 of the corrective insole.

Furthermore, a difference in height between the tip (a) and the heel (b) is made such that 60% to 80& of the weight of the body is scattered to the heel and 20% to 40% is scattered to the tip while the line of gravity passes between the calcaneus and the cuboid bone.

Here, if a person wears shoes having the corrective insoles of which the difference in height between the tip (a) and the heel (b) is 1 cm to 3 cm, because the tip (a) is slightly higher than the heel (b), it makes the center of gravity located between the calcaneus and the cuboid bone when the wearer stands up.

In the meantime, the bottom part 30 of the corrective insole 10 includes a plurality of protrusions 40 at the front side thereof for prevention of slipperiness, so that the insole does not slip inside the shoe.

Additionally, the corrective insole includes an arch part 100 rising on a surface part 20 so as to give a stimulus to a flat foot and to prevent a normal foot becomes a flat foot.

Now, actions of the corrective insole according to the present invention will be described as follows.

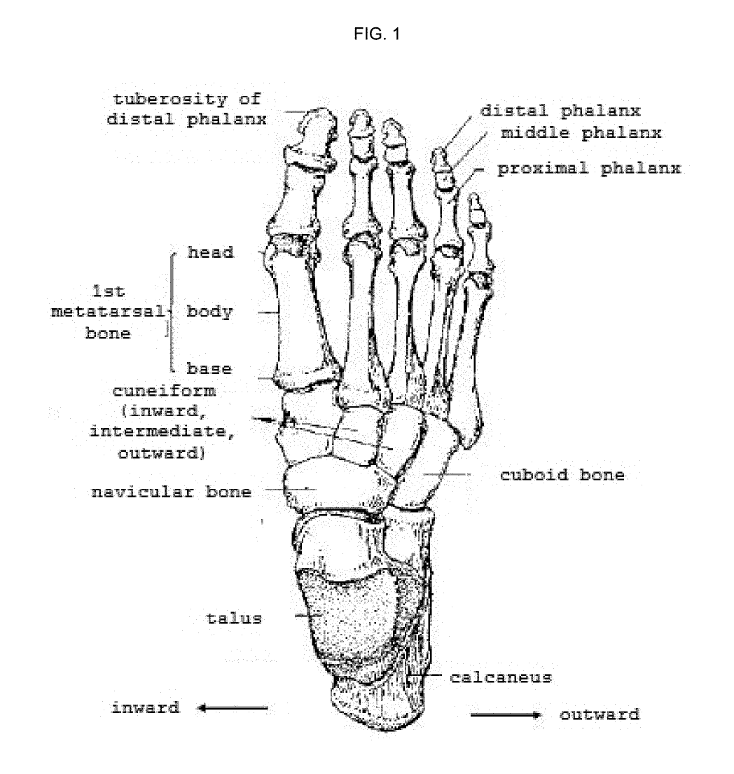

First, FIG. 1 is a view showing the structure of bones of the foot.

Here, the sole of a human body consists of two important arches: one being a lateral plantar arch for supporting the weight of the body; and the other being a medial plantar arch functioning as a spring during an exercise.

The lateral plantar arch includes the calcaneus which is the heel, the cuboid bone which is the most lateral of the bones, and the fifth metatarsal.

The medial plantar arch includes the calcaneus, the talus, the navicular, the cuneiform which is an arch part, and the first metatarsal bone.

However, the feet of the human body become flat feet due to exercises on a hard ground, sudden exercises, lack of flexibility in crural muscles, and so on.

In the meantime, because weight does not move to the lateral plantar arch while the weight moves from the outside to the inside in an arch form, load of the medial plantar arch cumulates, hence the arch form of the medial plantar arch is collapsed and the feet become flat feet.

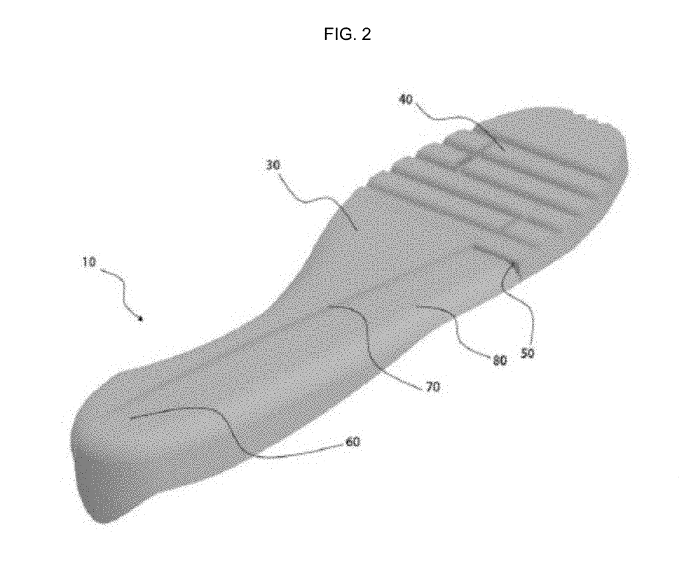

FIG. 2 is a perspective view showing a base side of a corrective insole according to a first preferred embodiment of the present invention.

Here, a bottom part 30 of the corrective insole 10 includes a posterior molded part 60 formed at the position corresponding to the calcaneus, an anterior height part 50 formed at the position corresponding to the head of the fifth metatarsal bone, and a skinned part 80 having a boundary protrusion part 70 formed between the posterior molded part 60 and the anterior height part 50.

Here, there is little difference in height between the tip (a) and the heel (b) or the tip (a) is slightly higher than the heel (b).

The corrective insole 10 of the present invention may be made through pressing or injection-molding using a mold.

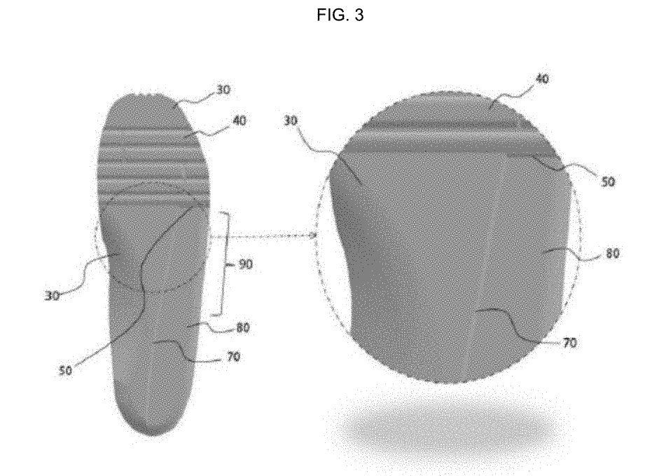

FIG. 3 is a front view showing the base side of the corrective insole according to the first preferred embodiment of the present invention

Here, the skinned part 80 makes weight shifted to the outside to scatter the weight along the most lateral side 90 of the corrective insole.

Furthermore, because there is little difference in height between the tip (a) and the heel (b) or the tip (a) is slightly higher than the heel (b), the skinned part 80 is made such that 60% to 80& of the weight of the body is scattered to the heel and 20% to 40% is scattered to the tip while the line of gravity passes between the calcaneus and the cuboid bone.

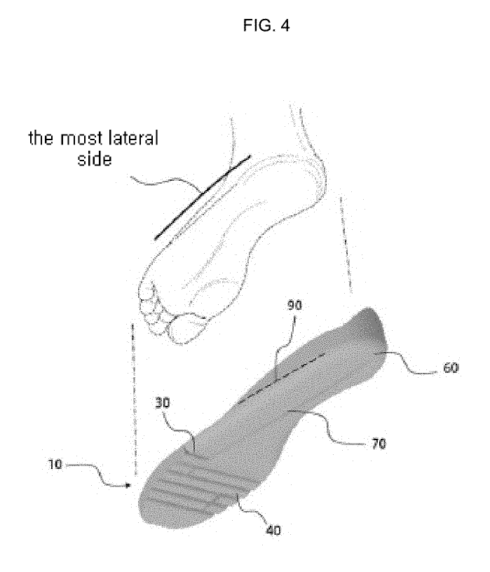

FIG. 4 is a schematic perspective view showing a used state of the corrective insole according to the first preferred embodiment of the present invention.

Here, if a person wears shoes (not shown) having the corrective insoles 10, the skinned part 80 makes the weight shifted onto the outside so as to scatter the weight along the most lateral side 90 of the corrective insole.

Moreover, if a person wears shoes (not shown) having the corrective insoles of which the difference in height between the tip (a) and the heel (b) is 1 cm to 3 cm, because the tip (a) is slightly higher than the heel (b), it makes the center of gravity located between the calcaneus and the cuboid bone when the wearer stands up.

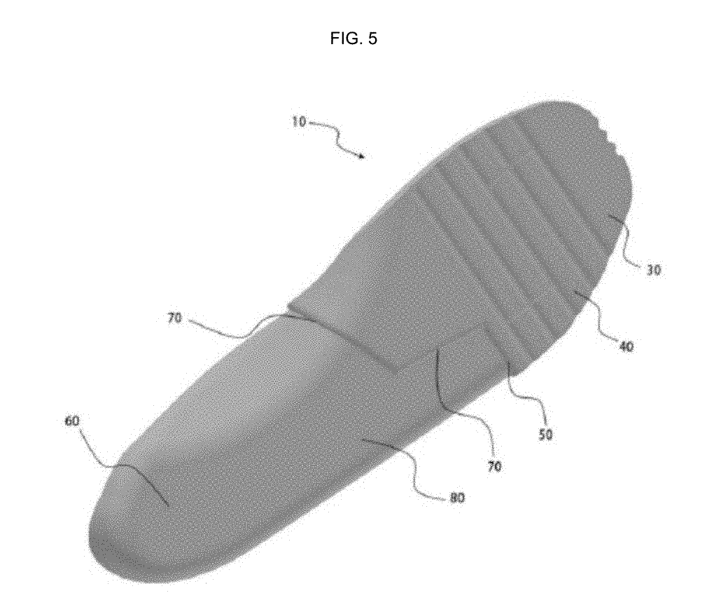

FIG. 5 is a perspective view showing a base side of a corrective insole according to a second preferred embodiment of the present invention.

Here, a bottom part 30 of the corrective insole 10 includes a posterior molded part 60 formed at the position corresponding to the calcaneus, an anterior height part 50 formed at the position corresponding to the head of the fifth metatarsal bone, and a skinned part 80 having a boundary protrusion part 70 formed between the posterior molded part 60 and the anterior height part 50.

The corrective insole 10 according to the present invention may be made through injection-molding using a mold.

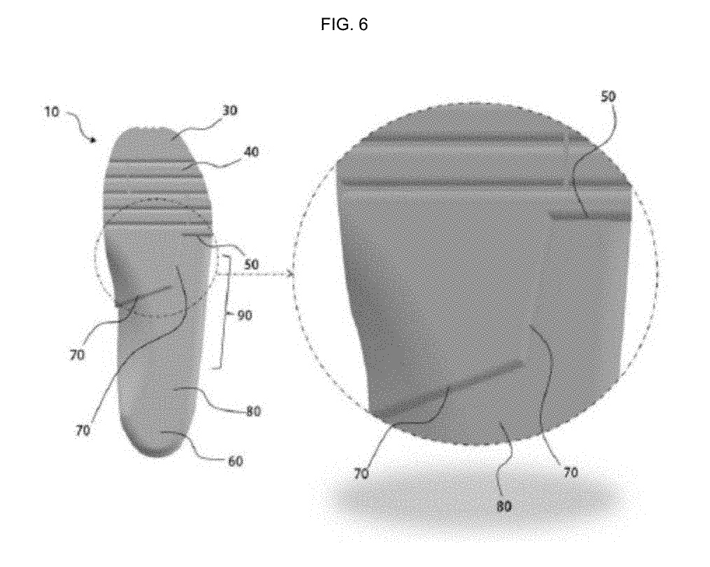

FIG. 6 is a front view showing the base side of the corrective insole according to the second preferred embodiment of the present invention.

Here, the skinned part 80 makes weight shifted to the outside to scatter the weight along the most lateral side 90 of the corrective insole.

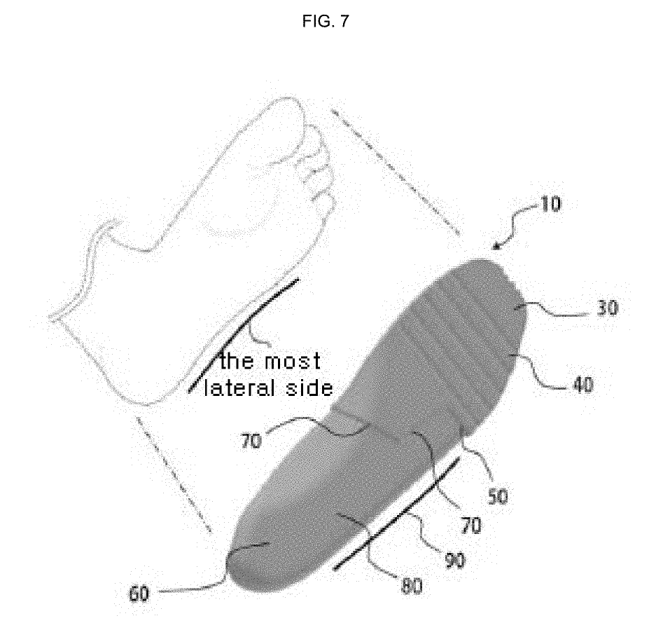

FIG. 7 is a schematic perspective view showing a used state of the corrective insole according to the second preferred embodiment of the present invention.

Furthermore, because there is little difference in height between the tip (a) and the heel (b) or the tip (a) is slightly higher than the heel (b), the skinned part 80 is made such that 60% to 80& of the weight of the body is scattered to the heel and 20% to 40% is scattered to the tip while the line of gravity passes between the calcaneus and the cuboid bone.

Here, the bottom part 30 of the corrective insole 10 includes a plurality of protrusions 40 at the front side thereof for prevention of slipperiness.

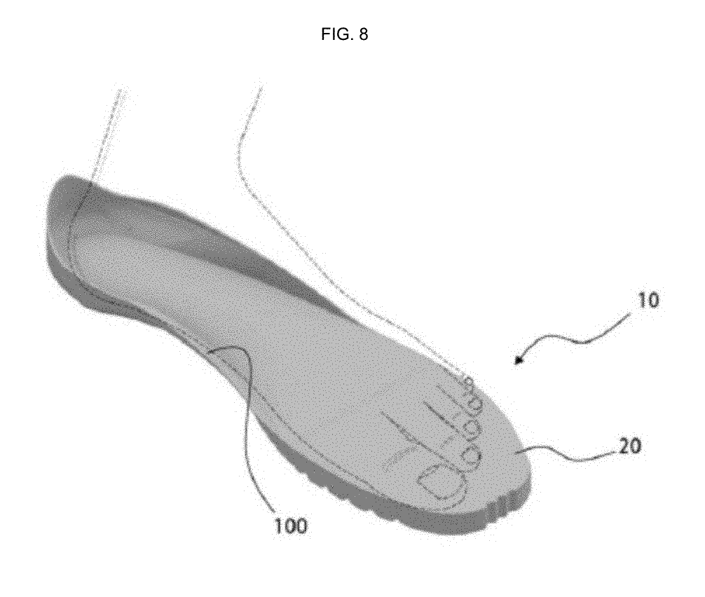

FIG. 8 is a perspective view showing a used state of the corrective insole of the present invention.

Here, a bottom part 30 of a corrective insole 10 according to a first preferred embodiment of the present invention has a skinned part 80 formed from the calcaneus to the head of the fifth metatarsal bone so that the weight of a human body can be easily transferred to the most lateral side 90 and can be scattered along the most lateral side 90.

In addition, the corrective insole includes an arch part 100 rising on a surface part 20 so as to give a stimulus to a flat foot and to prevent a normal foot becomes a flat foot.

In the meantime, in this specification, the corrective insole 10 which is configured as the lowermost component of a shoe is described, but if necessary, the present invention is applicable not only to an outsole but also to a midsole.

Moreover, of course, the corrective insole 10 is made of a buffering material with excellent strength and hardness.

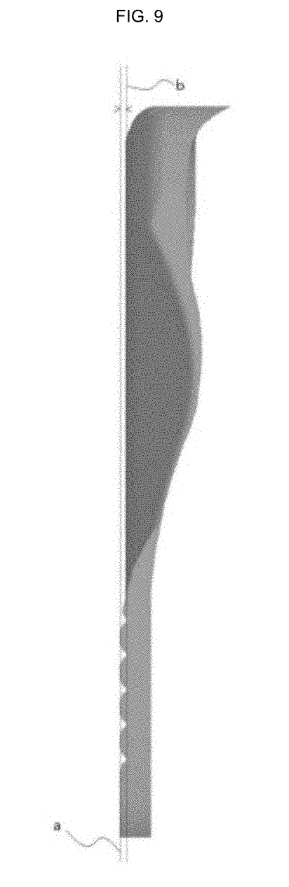

FIG. 9 is a front view showing a difference in height of the tip and the heel of the corrective insole according to the present invention.

As described above, the corrective insole according to the present invention makes a load by weight of the body and a load by an exercise shifted on the heel of the foot when the wearer stands up, and receives a load transferred from the calcaneus which is the heel of the foot and transfers the received load to the cuboid bone which is the most lateral of the bones of the foot, so as to optimally scatter the loads transferred to the foot.

Therefore, the corrective insole according to the present invention can prevent foot diseases and pronation, such as knock-kneed legs or bowed legs, and makes the load of the knee transferred from the anterior cartilage to the posterior cartilage through correction so that the pelvis, the back and the neck are straightened naturally.

The above description of the present disclosure is just for illustration, and a person skilled in the art will understand that the present disclosure can be easily modified in different ways without changing essential techniques or features of the present disclosure. Therefore, the above embodiments should be understood as being descriptive, not limitative.

For example, any component described as having an integrated form may be implemented in a distributed form, and any component described as having a distributed form may also be implemented in an integrated form.

The scope of the present disclosure is defined by the appended claims, rather than the above description, and ail changes or modifications derived from the meaning, scope and equivalents of the appended claims should be interpreted as falling within the scope of the present disclosure.

What is claimed is:

1. A corrective insole comprising a bottom part, the bottom part comprising:
a posterior molded part configured to be formed at a position corresponding to a calcaneus;
an anterior height part configured to be formed at a position corresponding to a head of a fifth metatarsal bone; and
a skinned part comprising a boundary protrusion part, which is formed between the posterior molded part and the anterior height part.

2. The corrective insole according to claim 1, wherein the skinned part is configured to shift a wearer's body weight to outside to scatter the wearer's body weight along a lateral side of the corrective insole.

3. The corrective insole according to claim 1, wherein the bottom part comprises a plurality of protrusions at a front side thereof to prevent slippage of the corrective insole inside a shoe.

4. The corrective insole according to claim 1, further comprising an arch part rising on a surface part of the corrective insole, the arch part being configured to provide a stimulus to a wearer's flat foot.

5. The corrective insole according to claim 1, further comprising an arch part rising on a surface part of the corrective insole, the arch part being configured to prevent flat foot.

6. A corrective insole comprising a skinned part configured to scatter 60% to 80% of a body weight of a wearer to a heel and 20% to 40% to a tip; and wherein the corrective insole is configured to have a minimal difference in height between the tip and the heel or the tip is configured to be higher than the heel such that a line of gravity passes between a calcaneus and a cuboid bone of the wearer.

7. The corrective insole according to claim 6, wherein the tip of corrective insole is configured to be 1 cm to 3 cm higher than the heel of the corrective insole such that a center of gravity is located between the calcaneus and the cuboid bone of the wearer in a standing position.
